(19) United States
(12) Patent Application Publication
(10) Pub. No.: US 2021/0124980 A1
(43) Pub. Date: Apr. 29, 2021

(54) AUGMENTED GROUP EXPERIENCE EVENT CORRELATION

(71) Applicant: Aetna Inc., Hartford, CT (US)

(72) Inventor: Robert E. Bates, III, Coupeville, WA (US)

(21) Appl. No.: 16/665,898

(22) Filed: Oct. 28, 2019

(51) Int. Cl. G06K 9/62; H04L 29/08; G06N 20/00

(52) U.S. Cl. CPC G06K 9/623; G06K 9/6262; G06N 20/00; G06K 9/6218; H04L 67/34; G06K 9/6257

(57) ABSTRACT

Systems and methods for capturing group experiences are described. Raw data logs from a plurality of user devices are received. Datasets from each raw data log are preprocessed. Dominant features for each dataset are extracted. The datasets are comparatively analyzed across raw data logs to determine common features in the datasets. The dominant and common features are then stored. Systems include wearable networks, user devices, a group event server and various datastores.

### BACKGROUND

Wearable sensors are an ever-increasing part of the daily human and animal interaction with the world. This is part of the larger movement of connected devices under the topical title Internet of things (IoT). IoT devices include electronics and any manner of connectivity for communication and interaction with or between other devices. IoT devices, including wearable sensors, have access both direct access to the internet and often through a gateway device and can be controlled remotely via the gateway device.

IoT devices include several professional applications but are increasingly becoming part of wireless sensor networks and control systems for home automation. Home automation or “smart home” devices such as lighting fixtures can be controlled via a home network, thermostats, cameras, refrigerators, and other home appliances. Adoption of IoT devices for increased connectivity with objects in the home is restricted to information about a particular home. For example, “smart home” devices for a specific individual's home operates as a standalone unit with limited connectivity to “smart home” devices of another person.

Furthermore, with different devices having different manufacturers and existing within different ecosystems, even IoT devices within a specific individual's “smart home” environment may have limited communication. For example, a wearable exercise tracker with a heart rate monitor connected to a father's mobile phone and a different exercise tracker is tied to his son's phone. With different phones, different exercise trackers, and distinct permissions, there is little, or no information the personal wearable devices can share and even less can be discerned about the potential combined experience of the father and the son are in the same vicinity enjoying a day out together. Information from the father's mobile phone and relayed information from the exercise tracker is distinct. The son's mobile phone is also distinct. Some basic comparative data could be shared through a share application; no meaningful conclusions can be drawn from the experience between the father and the son from the siloed data collected by both the father's and the son's mobile phones.

### SUMMARY

In one an embodiment, a method for capturing group experiences are provided. The method is performed by connected wearable IoT devices, a network gateway(s) device, a server(s) each comprised of microprocessors, processors and memory storage. The method includes receiving raw data logs from a plurality of user devices, a respective raw data log corresponding to a respective user device. Datasets from each raw data log are preprocessed. Dominant features for each dataset are extracted. The datasets are comparatively analyzed across raw data logs to determine common features in the datasets. The dominant and common features are then stored.

In another embodiment, an event computing node(s) or server including a processor and a non-transitory computer-readable medium storing instructions, that when executed by the processor, cause the processor to perform steps is provided. The steps include receiving raw data logs from a plurality of user devices, a respective raw data log corresponding to a respective user device. Datasets from each raw data log are preprocessed. Dominant features for each dataset are extracted. The datasets are comparatively analyzed across raw data logs to determine common features in the datasets. The dominant and common features are then stored.

In yet another embodiment, a non-transitory computer-readable medium storing instructions, that when executed by a processor, cause the processor to perform steps is provided. The steps include receiving raw data logs from a plurality of user devices, a respective raw data log corresponding to a respective user device. Datasets from each raw data log are preprocessed. Dominant features for each dataset are extracted. The datasets are comparatively analyzed across raw data logs to determine common features in the datasets. The dominant and common features are then stored.

### BRIEF DESCRIPTION OF THE DRAWINGS

FIG. 1 illustrates a system for group experience event correlation according to an embodiment of the disclosure;

FIG. 2 illustrates a computing device according to an embodiment of the disclosure;

FIG. 3 is a flow diagram illustrating a process for ingesting raw data according to an embodiment of the disclosure;

FIG. 4 is a flow diagram illustrating a process for determining learned features and providing output recommendations to group participants, according to an embodiment of the disclosure;

FIG. 5 illustrates a system for group experience event correlation according to an embodiment of the disclosure;

FIG. 6 illustrates a system for group experience event correlation according to an embodiment of the disclosure; and

FIG. 7 is a flow diagram illustrating a process for monitoring a group experience event according to an embodiment of the disclosure.

### DETAILED DESCRIPTION

Utilization of wearable tech for self-quantification and analysis is increasingly common place, so embodiments of the disclosure provide systems and methods for using sensor technology to capture, store, analyze, and cause actions based on triggers produced by a group of individuals. The embodiments can be used in a variety of group interactions where emotional and experiential changes can influence immediate augmented experiences or provide inputs for the optimization of future experiences.

There are several problems that can be envisioned in group activities or experiences which embodiments of the disclosure can alleviate. Firstly, technological interruptions can distract from flow of an experience; these interruptions, whether major or minor, can be invasive and can interrupt participation in group experiences with families and close relationships (professional or personal). For example, in the flow of experience, stopping to state, “everyone wait, I have to open my phone camera app” before taking a photo can take away from the group experiences.

Secondly, group dynamics present expressive challenges in close family or relationship clusters. Often dominant or stronger personalities drive activity or conversation. Embodiments of the disclosure can use sensors, preferences and aggregate experience to determine whether group satisfaction or dissatisfaction is understood by everyone in the group. Embodiments of the disclosure can also provide how to potentially create a better or optimal experience for all within the group or how to create experiences which address individual wants in a group dynamic

Thirdly, group personalization sometimes does not complement individual personalization contextualization. In society, personalization is often linked to a payor—the one who spends money—and not to the group who are the benefactors of the payor. Embodiments of the disclosure can draw a distinction and provide details around personalization of individuals participating in a group activity which may not have participated in consumer transactions leading up to the group activity.

Embodiments of the disclosure provide multi-event or personalized triggers in a group environment. Automation of any process often creates cascading actions or events either with one individual or with multiple individuals who are not participating in the same activity, location, and context. By collecting sensor data, preferences, and getting real-time feedback, highly-personalized timely actions can be automated in a shared context—location and activity.

Embodiments of the disclosure provide insight into the real-time effect on the group and individual health. Wearables often measure activity tracking but are not tuned to measure comprehensive health—both physical and emotional. Through a networked-awareness of one or more sensors on an individual with the network of other persons' linked wearables within the context of the activity being measured, trend or indicative effects on emotional health can be measured as well as the variation in the exertion levels by individuals participating in a group activity. The individuals can share collected activity data, and for a relatively common task, differential measurements on physical impact can be used to determine the relative trend on comprehensive health over time.

Embodiments of the disclosure can improve business and collaboration performance Verbal expression is often limited by time and dominance of persona in a collaboration or business setting. Dominance can be through personality or social or corporate hierarchy. Using networked sensors, preferences and historical data, positive and negative nonverbal responses can be gathered using the variations in the human physiology. This could be incredibly important in driving strategic changes, large initiatives and much more within a task and activity-dependent organization.

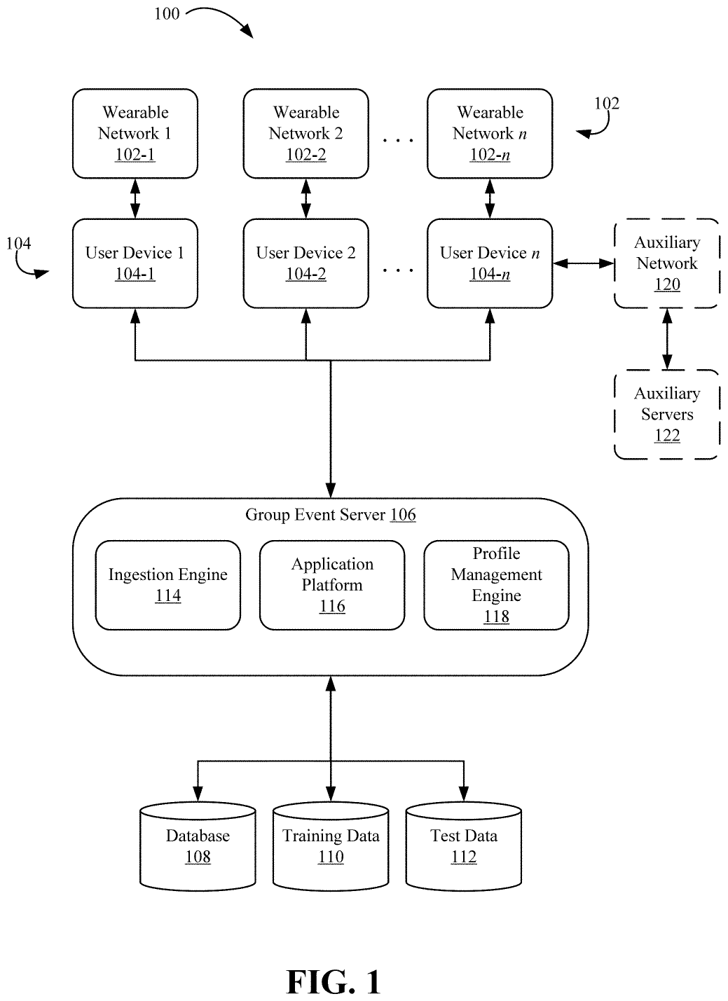

FIG. 1 illustrates a system 100 for group experience event correlation according to an embodiment of the disclosure. The system 100 includes two or more wearable networks 102 identified as wearable network 1 102-1, wearable network 2 102-2, . . . , wearable network n 102-n; two or more user devices 104 identified as user device 1 104-1, user device 2 104-2, . . . , user device n 1 104-n; a group event server 106; and one or more and storage devices or databases, e.g., database 108, training data 110, and test data 112. In some embodiments, the system 100 can include an auxiliary network 120 and auxiliary servers 122.

The wearable networks 102 include one or more wearable devices, IoT devices, or sensors, e.g., body cameras, clothing cameras, fitness trackers, haptic devices, heart rate monitors, optical sensors, temperature sensors, accelerometers and other inertial sensors, smart glasses, smart watches, chemical and biological sensors embedded in clothing, and so on. Each wearable network 102 is associated with a user device 104. As depicted in system 100, wearable network 1 102-1 associates with user device 1 104-1. The user device 1 104-1 serves as a hub for collecting and aggregating information collected from the wearable network 1 102-1.

The user devices 102, the group event server 106, the auxiliary network 120, the auxiliary servers 122, and the various databases, e.g., the database 108, the training data 110, and the test data 112 are computing devices with a processor and a non-transitory computer-readable medium. The user device 102 can be a desktop computer, a laptop computer, a smartphone, a smartwatch, a smart television, a router, and so on.

The group event server 106, the auxiliary servers 122, and the various databases can be realized by a cloud service, a server, and so on. The group event server 106 implements several software engines that cooperate to determine group dynamics given specific event related data obtained from the user devices 104. A software engine is a combination of hardware and software for performing one or more specific functions. The group event server 106 includes an ingestion engine 114 for preprocessing or processing raw data obtained from the user devices 104, an application platform 116 for providing feedback to the user devices 104 and for managing learned data characteristics in the raw data, and a profile management engine 118 for customizing user preferences and defining groups.

The database 108 is provided for storage of learned data characteristics and other data generated by the group event server 106. The database 108 can also include parameters for various machine learning algorithms In some embodiments, the training data 110 and the test data 112 are contained in the database 108.

The auxiliary network 120 is a third party network/device, e.g., a smart speaker at home not part of the wearable networks 102, or an interactive sensor at a supermarket or a store. The auxiliary network 120 provides additional information to the user devices 104. The auxiliary network 120 can be served by the auxiliary servers 122. The auxiliary servers 122 can include storage and other informatics to support functions of the auxiliary network 120.

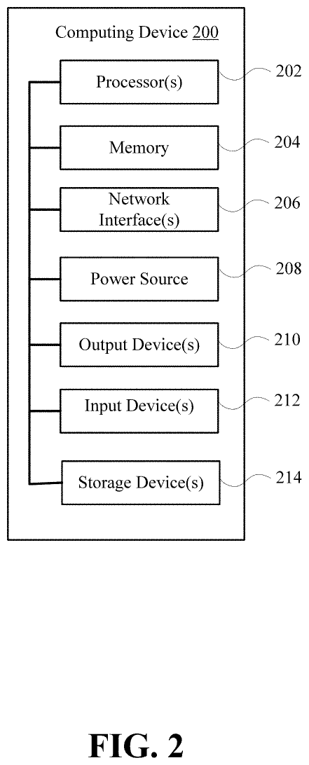

FIG. 2 is a block diagram illustrating basic hardware components of a computing device 200, according to some example embodiments. The computing device 200 is an architecture for one or more servers for implementing the auxiliary network 120, the auxiliary servers 122, the group event server 106 and the different databases. The computing device 200 also is an architecture for the user device 104 and some wearable devices within the wearable networks 102. The computing device 200 may include one or more processors 202, memory 204, network interfaces 206, power source 208, output devices 210, input devices 212, and storage devices 214. To simplify the discussion, the singular form will be used for all components identified in FIG. 2, when appropriate, but the use of the singular does not limit the discussion to only one of each component. For example, multiple processors may implement functionality attributed to processor 202.

Processor 202 is configured to implement functions and/or process instructions for execution within the computing device 200. For example, processor 202 executes instructions stored in memory 204 or instructions stored on a storage device 214. In certain embodiments, instructions stored on storage device 214 are transferred to memory 204 for execution at processor 202. Memory 204, which may be a non-transient, computer-readable storage medium, is configured to store information within the device 200 during operation. In some embodiments, memory 204 includes volatile memories such as RAM, dynamic random access memories (DRAM), and static random access memories (SRAM). Memory 204 also maintains program instructions for execution by the processor 202 and serves as a conduit for other storage devices (internal or external) coupled to the computing device 200 to gain access to processor 202.

Storage device 214 includes one or more non-transient computer-readable storage media configured for long-term storage of information. In some embodiments, the storage device 214 includes floppy discs, flash memories, magnetic hard discs, optical discs, solid-state drives, or forms of electrically programmable memories (EPROM) or electrically erasable and programmable (EEPROM) memories.

Network interfaces 206 are used to communicate with external devices and/or servers. The computing device 200 may include multiple network interfaces 206 to facilitate communication via multiple types of networks. Network interfaces 206 may include network interface cards, such as Ethernet cards, optical transceivers, radio frequency transceivers, or any other type of device that can send and receive information. Examples of network interfaces 206 include radios compatible with several Wi-Fi standards, 3G, 4G, Long-Term Evolution (LTE), Bluetooth®, etc.

Power source 208 provides power to the computing device 200. Examples include rechargeable or non-rechargeable batteries utilizing nickel-cadmium or other suitable material. Power source 208 may include a regulator for regulating power from the power grid in the case of a device plugged into a wall outlet.

The computing device 200 may also be equipped with one or more output devices 210. Output device 210 is configured to provide output to a user using tactile, audio, and/or video information. Examples of output device 210 may include a display (cathode ray tube (CRT) display, liquid crystal display (LCD) display, LCD/light-emitting diode (LED) display, organic LED display, etc.), a sound card, a video graphics adapter card, speakers, magnetics, or any other type of device that may generate an output intelligible to a user of the computing device 200.

The computing device 200 may also be equipped with one or more input devices 212. Input devices 212 are configured to receive input from a user or the environment where the computing device 200 resides. In certain instances, input devices 212 include devices that provide interaction with the environment through tactile, audio, and/or video feedback. These may include a presence-sensitive screen or a touch-sensitive screen, a mouse, a keyboard, a video camera, a microphone, a voice responsive system, or any other type of input device.

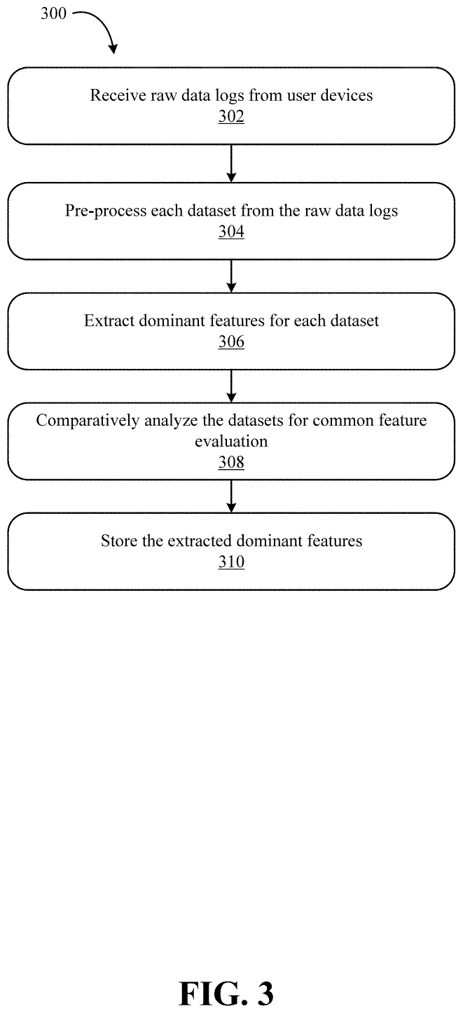

FIG. 3 is a flow diagram illustrating a process 300 performed by the group event server 106, for ingesting raw data from the user devices 104, according to an embodiment of the disclosure. The process 300 can be performed by the ingestion engine 114 of the group event server 106. At step 302, the ingestion engine 114 of the group event server 106 receives raw data logs from the user devices 104. In an example, user device 1 104-1, user device 2 104-2, and user device 3 104-3 provide raw data logs to the group event server 106. Each raw data log provided by the user devices 104 can include multiple types of data, e.g., heart rate measurements, geolocation data, and body temperature measurements. Each type of data in the raw data logs is updated at a regular frequency such that each raw data log includes at least two data points for each type of data. For example, there are at least two measurements of heart rate in the raw data log, and at least two different probings of geolocation data even if the location is the same for both probing. The raw data logs are created based on information obtained by the user devices 104 through the wearable networks 102, and in some cases, the auxiliary network 120.

At step 304, the ingestion engine 114 of the group event server 106 pre-processes each dataset from the raw data logs of step 302. For a respective raw data log, each dataset in the log is pre-processed. Pre-processing can include data cleansing, substitution, imputation, standardization, and so on. For example, heart rate measurements can be provided at intervals as beats per minute, geolocation data can be provided at intervals as coordinates, and body temperature can be provided as average body temperature for specific intervals. In some instances wearable networks 102 can miss a measurement or data may be noisy, so the ingestion engine 114 pre-processes each dataset received for each type of data.

At step 306, the ingestion engine 114 of the group event server 106 extracts dominant features for each dataset. For a specific wearable network, e.g., wearable network 1 102-1, the ingestion engine 114 looks for feature definitions for each dataset. For example, during a specified period if electrodermal activity determines changes in heartrate, skin conductance (perspiration), sensors determine body temperature changes, accelerometers and pedometers detect increased movement of the body and limbs of an individual altered significantly from baseline, the ingestion engine 114 determines that one of these measurements variance from baseline may indicate a notable dataset feature or in this case “an event” may have occurred and notes the geolocation during that specific period. An example of a relevant event is a participant using the user device 1 102-1 may be excited, or the participant's emotions may be elevated. Features in the data, independent measurable variables, at this point, are noted. While the most dominant features are noted, these features are not yet deterministic until compared with other participants. Data set features could also come from other sensors or content for participants in the group event. For example, features might come from direct measurement of wearable sensors for analytical review of the content generated during a potential event. Wearable sensors include but are not limited to digital cameras, thermometers, accelerometers, pedometers, heart rate monitors, altimeters, barometers, compasses, GPS receivers, and electrodermal activity. Analytical insights on content generated during events might include pupil dilation, facial expression recognition, as well as to object recognition in background or foreground photos and videos.

At step 308, the ingestion engine 114 of the group event server 106 comparatively analyzes the datasets for all participants for common feature evaluation. The ingestion engine 114 takes specific features from step 306 to see whether there are common features among datasets obtained from the user devices 104. For example, the user device 1 104-1 provided data that suggested that its participant is excited, and the user device 2 104-2 also provided data that suggested that its participant is excited. The excitement feature present in datasets from both the user device 1 104-1 and the user device 2 104-2 is a common feature.

At step 310, the ingestion engine 114 of the group event server 106 stores the extracted dominant features in the database 108.

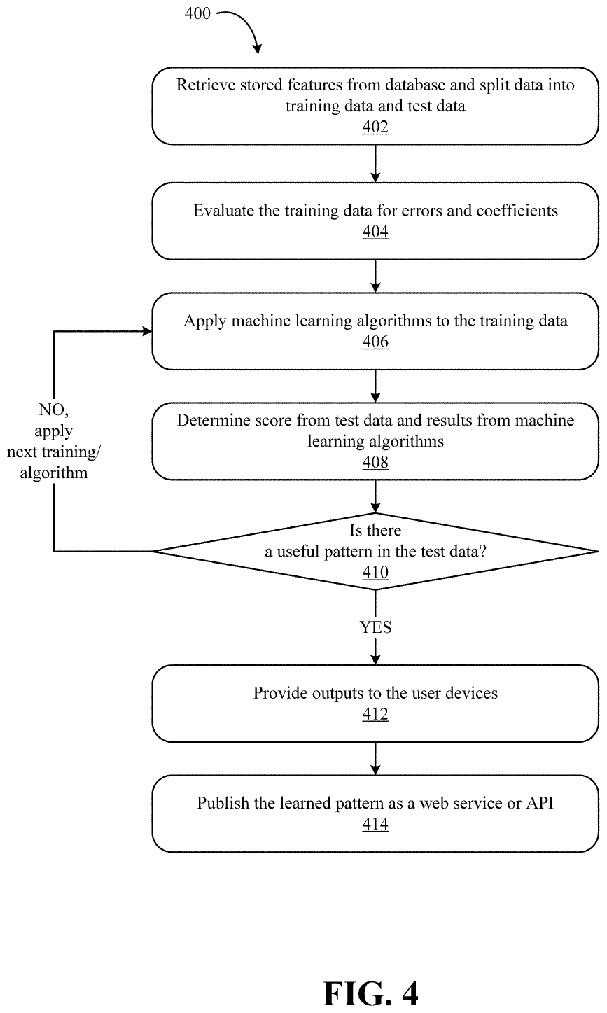

FIG. 4 is a flow diagram illustrating a process 400 performed by the group event server 106, for determining learned features and providing output recommendations to group participants, according to an embodiment of the disclosure. At step 402, the group event server 106 retrieves stored features from the database 108 and splits data into training data 110 and test data 112. Test data may yield statistically meaningful results on its own and may possess the characteristics of data to be measured. Training data, which represents characteristics of the overall data, is regularly captured and stored in the system for future machine learning training.

At step 404, the group event server 106 evaluates the training data 110 for errors and coefficients. Data is evaluated for accuracy of predictions. How often predictions are incorrect is recorded as classification errors. Training errors and test errors are determined.

At step 406, the group event server 106 applies machine learning algorithms to the training data 110. Machine learning can be supervised or unsupervised, depending on the objectives of the training. The initial primary approach will be supervised where the training data is used to identify relevant events or arrive at relatively accurate conclusions. Example of machine learning algorithms might include regression learning systems which look for sustained periods of anomalies outside of predictable measurement for an individual.

At step 408, the group event server 106 determines a score from the test data 112 and results from the machine learning algorithm applied at step 406. Sample test and scoring options include cross-validation and percentage split.

At 410, the group event server 106 determines whether there is a useful pattern in the test data 112. In an example where heartrate, geolocation, and body temperature are collected, a useful pattern may be that the group event server 106 observes that a correlated rise in heart rate and body temperature over a specific period seem to be correlated with specific geolocation. This can represent a sample of a data feature which becomes increasing contextual if similar responses are measured in other persons in the same network who are also near or at the same geolocation.

If there is no useful pattern in the test data 112, then a new machine-learning algorithm not yet applied is chosen, and step 406 is performed. If there is a useful pattern in the test data 112, then at step 412, the application platform 116 of the group event server 106 provides outputs to the user devices 104. An example of the output can include providing to a specific group of user devices 104, an alert that during a time interval at a specific location, the participants' body temperature and heart rate rose which indicates that all or most individuals in the group were excited. Also, if certain participants were in the minority and did not exhibit the observed trend, then the group event server 106 can provide a message inquiring whether the individual is doing well.

At step 414, the application platform 116 of the group event server 106 publishes the learned pattern as a web service or application programming interface (API). In an example where heartrate, geolocation, and body temperature are collected, a learned pattern where geolocation is associated with a rise in heart rate and body temperature can be published. The published pattern allows the group event server 106 to look for this potential pattern in future events.

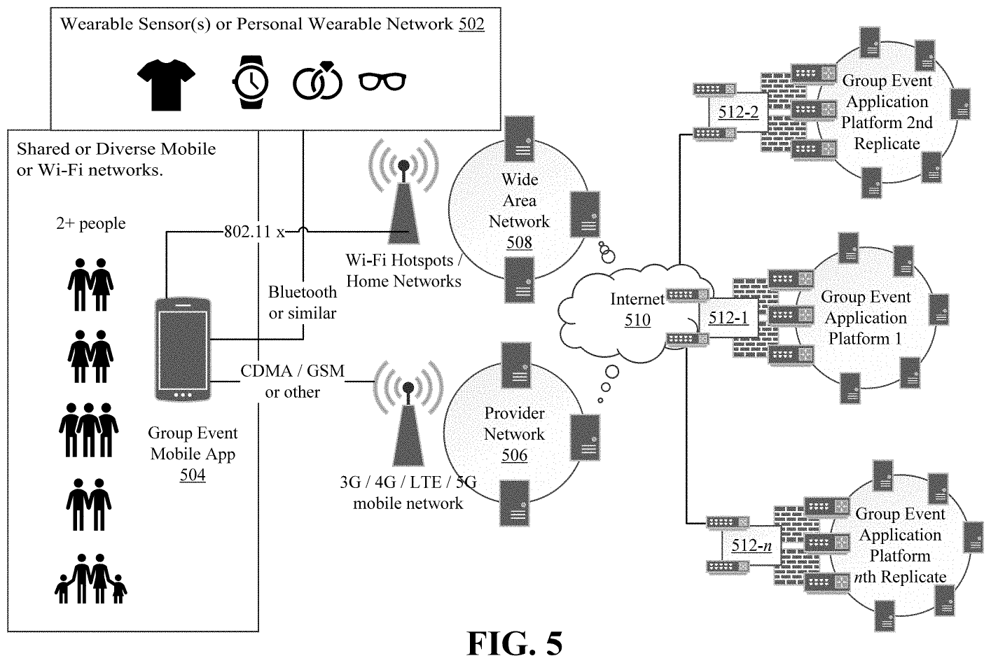

FIG. 5 illustrates a system for group experience event correlation according to an embodiment of the disclosure. In FIG. 5, each participant or member of a group is equipped with wearable sensors or a personal wearable network 502. Each participant is also equipped with a group event mobile app 504, running on a mobile phone. The personal wearable network 502 for each member can communicate with a respective group event mobile app 504 via Bluetooth® or some other shorter range wireless protocol.

The group event mobile app 504 can connect to the internet 510 via a Wi-Fi or home network using an 802.11x protocol and/or via a mobile network technology which includes code division multiple access (CDMA) or global system for mobile communications (GSM). A home network connection to the internet 510 is indicated as wide area network 508, and a mobile network connection to the internet 510 is indicated as provider network 506.

Via the internet 510, the group event mobile app 504 can communicate with a group event application platform running in a cloud or server. The group event application platform in FIG. 5 is an example of the application platform 116 of the group event server 106 of FIG. 1. Multiple replicates of the group event application platform may be running in the cloud such that load balancers and firewalls 512 refer the group event mobile app 504 to a specific replicate.

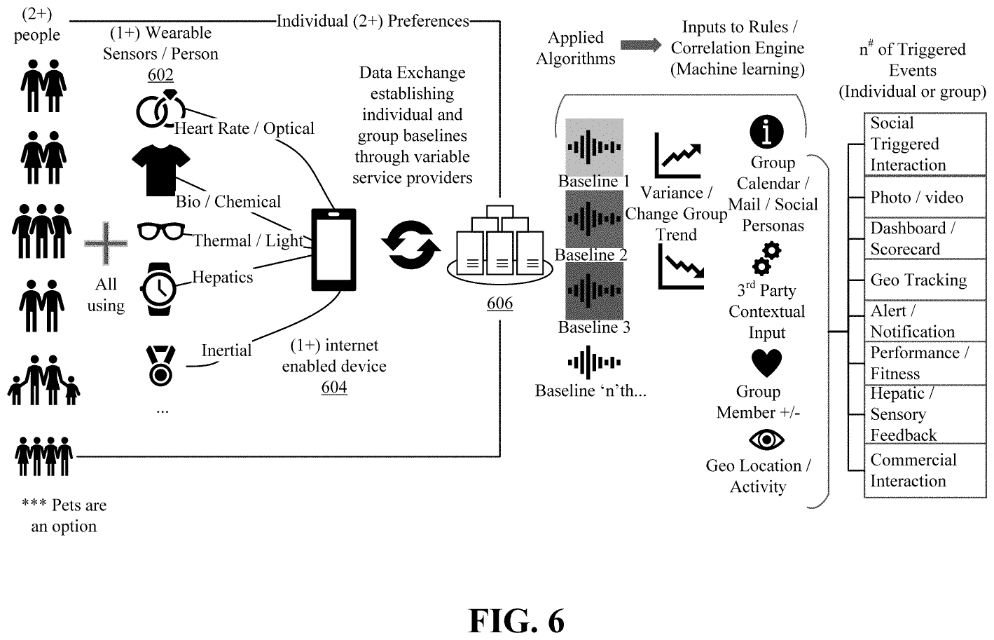

FIG. 6 illustrates a system for group experience event correlation according to an embodiment of the disclosure. In FIG. 6, each participant or member of a group is equipped with wearable sensors 602 and an internet-enabled device 604. The internet-enabled device 604 can be a mobile phone and is configured to communicate with a group event server 606 according to some embodiments of the disclosure. The group event severs 606 can be, e.g., the group event server 106 with a profile management engine 118.

The group event server 606 can store member preferences in its profile management engine. The group event server 606 also determines member baselines according to the ingestion process of FIG. 3 and the learned patterns determined via the process of FIG. 4. The group event server 606 can use the baseline of the different members to determine how each individual's baseline is affecting the group. The group event server 606 can determine variance and changes within the group trend and perform certain actions based on the determined variance. The group event server 606 may have access to group calendar or mail, and geolocation activity via the internet-enabled device 604. The group event server 606 can also receive third-party contextual input. Example of third party inputs might include targeted messaging for groups having a particular dining experience after a shared event where they were physically active. Once a group pattern is understood, future alternative groups who have similar preferences, activities, and/or locations can be identified. Correlated emotional responses may assist in further identifying alternative groups.

Personalized profiles or personas are managed by the profile management engine 118. The wearable sensors 602 can create a wireless body area network to give simple to complete data on the current physical and interpreted the emotional state of a participant or member. This data is used to build a baseline for a ‘normal’ state—emotionally stable or comfortable, and physical activity is within an average range within geography frequently traveled by the member. Persona or profile data is then used to determine experience criteria when combined with variances to baseline data.

The group event server 606 can perform several actions based on correlation thresholds for a member or the group being triggered. Actions that can be triggered include the group event server 606, sending a message to one or more internet-enabled devices 604 encouraging social interaction. The group event server 606 an send a message to one or more internet-enabled devices 604 to commence photo or video capture either using a camera on the internet-enabled device 604 or using one of the wearable sensors 602. The group event server 606 can provide a dashboard or scorecard with analytics showing an interval where members of the group had a certain experience. The group event server 606 can trigger geo-tracking of members of the group. The group event server 606 can alert and notify members of the group when an event is triggered. The group event server 606 can provide performance or fitness metrics such as average heart rate change, group calories estimated burned, or floors climbed as a group. The group event server 606 can provide haptic and/or sensory feedback alerting individuals or the group of optimal shared moments. Hepatic patterns, vibrating pulses, or audible sounds could be used to create subtle alerts in various personal network devices or be used as a mechanism to alert all group members or an individual of an opportunity or action to be taken. The group event server 606 can interact with members at specific times determined that they may be interested in a commercial activity.

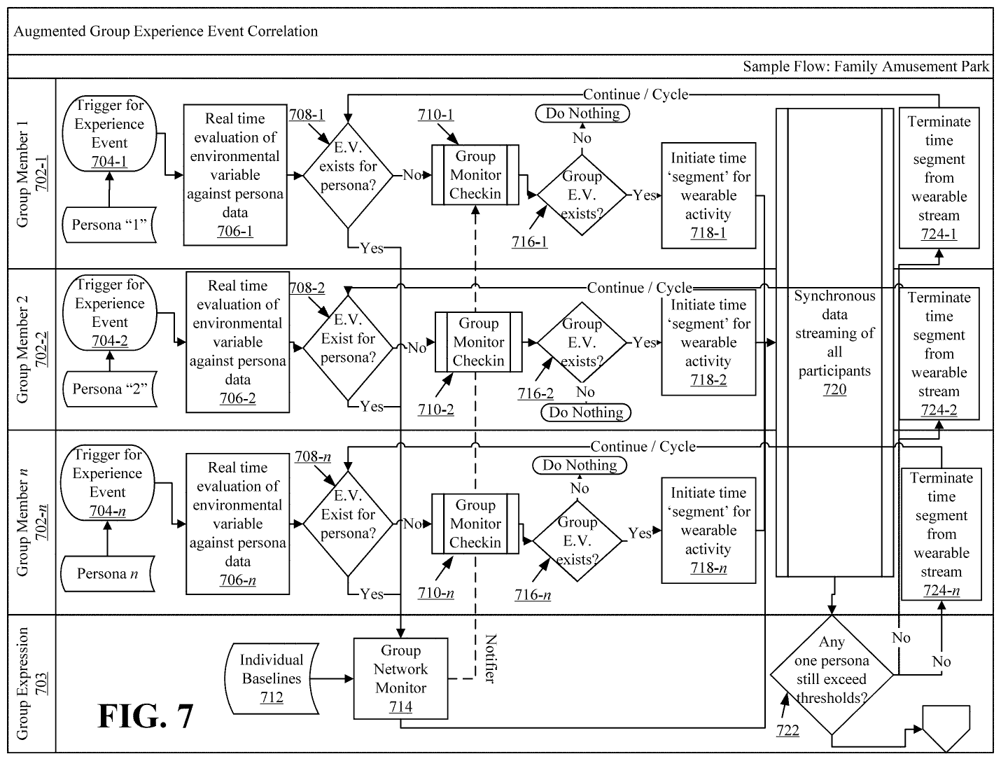

FIG. 7 is a flow diagram illustrating a process for monitoring a group experience event according to an embodiment of the disclosure. FIG. 7 provides an example where a family is participating in an amusement park adventure. Each group member 702 has a wearable network for collecting sensory data and a profile or persona stored on a group event server according to embodiments of the disclosure. There are n group members, and the group event server performs the process for monitoring the group experience event in FIG. 7 for each group member.

At step 712, the group event server, an assigned server or cluster of servers used for collecting data on the entire group, monitors individual baselines of each member of the group. At step 714, the individual baselines of each member inform a group network monitor.

At step 704, the group event server, after identifying a correlated event for one or more members of the participating group, generates a trigger for an experienced event based on profile data or persona data of at least one group member 702. For example, when several members of an enrolled group are visiting an amusement park together, a trigger may be generated. While all members of a group participate in a variety of rides, games, and activities, triggers are generated when group heart rates are elevated, gyroscopic sensors track more than baseline motions, and audio samples show increased laughter all occur together amongst members of the enrolled group. These correlated events create correlated triggers. These correlated triggers can precipitate events where the group records key photos or videos, receives special incentives aligned to personal profiles and interests

At step 706, the group event server evaluates environmental variables (E.V.) against persona data in real-time after the trigger is generated. The most common environmental variable correlated with triggers is geolocation. Alternate environmental variables could include ambient temperature, weather conditions, and seasonal time of year. All of these environmental variables can be used to enhance the effective use of triggers.

At step 708, the group event server determines for each member of the group whether the E.V. exists.

At step 710, if the E.V. exists for a specific persona, then a group monitor checking is performed. At step 716, if the E.V. does not exist for the group, then the group event server does nothing. Otherwise, if the E.V. exists for the group, at step 718, the group event server initiates a time segment for wearable activity. Again, using the amusement park as a setting for a group activity. While exploring the amusement park, one member of the group may choose to play a carnival or video game. While the one member plays a game, their personal network sensors are engaged, but the other members of the group's sensors stay at or near the baseline. Therefore, the group event server(s) detects that this is a non-event and no trigger is generated.

At step 720, the group event server captures or streams data of all members synchronously to determine at step 722 whether any one persona still exceeds thresholds. If after a certain interval of capturing or streaming data, when a persona no longer exceeds thresholds, then at 724, the group event server marks a terminate time segment from the wearable stream.

In performing the process provided in FIG. 7, the group E.V. being monitored is used to start a data streaming process, and the individual E.V. being monitored is used to stop the data streaming

Embodiments of the disclosure provide a system and method for collecting contextual data as participating individuals move through space (various geo locations) over time. Examples of data to be collected might include heart rate, body temperature, gyroscopic orientation, altitude, voice/audio, video/digital photographic, and so on. Data collected comes from a personal wearable network worn by each participating member in the group as well as any proximal sensors or auxiliary networks which the individuals have access to or are enrolled with as a service. Incremental data collection, while participants move through various locations together describes a data collection approach over time, which can be used to define common features between datasets used to identify shared experiences. This incremental data collection approach can be called a “bread crumb” data collection approach.

Personal wearable network devices, worn by participating individuals, are each authorized to connect to a personal mobile device via Bluetooth or similar limited area network technology. Each device provides a continuous stream of data to the mobile device with individual sensor data specific to each participating device in the wearable network. Devices in this network might include but are not limited to watches, necklaces, rings, glasses, and much more. Data from each device is stored on the mobile device according to its native integration or application. Embodiments of the disclosure also allow data storage to occur over a common timeline.

In some embodiments, participants can individually or commonly authorize data or sensory collection of information by local or proximal sensors of an auxiliary network. Proximal sensors can provide additional data streams for motion while indoors, ambient temperature, content on display, and audio or video recordings. If these devices have similar local connectivity to the personal wearable network, full data streaming to the individual can occur. If full data streaming is not available or connectivity is unstable, a shorter key or hash representation can be communicated to the individual's phone for future access and data integration by the group event server. The group event server having authorized access to proximal sensor data can then overlay the proximal sensor data on the same timeline with personal network data for feature identification.

If communication with the phone is not possible, the group event server can regularly poll authorized proximal sensor subscriptions for data streams triggered via mobile device registered locations. These data ‘bread crumbs’ represent the coordinate trail of data collection and interactions between participating members of the group. Individual and group data features are identified post data cleansing.

By relying on data bread crumbs, embodiments of the disclosure can effect a faster machine learning process. Clusters of individuals at the same location at roughly the same time reduces the need for analyzing a large dataset at the get-go which conventional machine learning approaches rely upon. Using individuals' self-clustering through groups, the group event server can learn patterns much quicker and perform sentiment analysis on a smaller set of data than conventional methods.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for capturing group experiences, the method performed by a server comprising a processor and memory, the method comprising:
receiving raw data logs from a plurality of user devices, a respective raw data log corresponding to a respective user device;
preprocessing datasets from each raw data log;
extracting dominant features for each dataset;
comparatively analyzing the datasets across raw data logs to determine common features in the datasets; and
storing the dominant and common features.

2. The method according to claim 1, wherein each raw data log comprises:
heartrate measurements, geolocation data, and body temperature measurements.

3. The method according to claim 1, wherein preprocessing the datasets comprises performing one or more of:
data cleansing and substitution.

4. The method according to claim 1, wherein dominant features for a first dataset is determined based on a correlation between the first dataset and a second dataset.

5. The method according to claim 4, wherein each raw data log includes geolocation data and after extracting the dominant features for the first dataset, the dominant feature for the first dataset is marked with the geolocation data.

6. The method according to claim 1, further comprising:
retrieving the stored features from the database and splitting data from the stored features into training data and test data;
evaluating the training data for errors and coefficients;
applying machine learning algorithms to the training data; and
determining a score from the test data and machine learning algorithm results of the training data.

7. The method according to claim 6, further comprising:
based on the score, determining that there is a useful pattern in the test data; and
providing outputs to the user devices.

8. The method according to claim 7, further comprising:
publishing the useful pattern as a web service or an application programming interface (API).

9. The method according to claim 7, wherein providing the outputs to the user devices comprises one or more selected from the group consisting of:
providing a dashboard or scorecard to the user devices;
providing a signal to the user devices to initiate a photo or video capture;
providing a message to the user devices, the message comprising recommendations for a group event; and
providing a product recommendation message to the user devices.

10. The method according to claim 6, further comprising:
based on the score, determining that there is no useful pattern in the test data; and
applying a different machine learning algorithm to the training data.

11. An event server comprising:
a processor; and
a non-transitory computer-readable medium storing instructions, that when executed by the processor, cause the processor to perform steps including:
receiving raw data logs from a plurality of user devices, a respective raw data log corresponding to a respective user device;
preprocessing datasets from each raw data log;
extracting dominant features for each dataset;
comparatively analyzing the datasets across raw data logs to determine common features in the datasets; and
storing the dominant and common features.

12. The event server according to claim 11, wherein each raw data log comprises:
heartrate measurements, geolocation data, and body temperature measurements.

13. The event server according to claim 11, wherein preprocessing the datasets comprises performing one or more of:
data cleansing and substitution.

14. The event server according to claim 11, wherein dominant features for a first dataset is determined based on a correlation between the first dataset and a second dataset.

15. The event server according to claim 14, wherein each raw data log includes geolocation data and after extracting the dominant features for the first dataset, the dominant feature for the first dataset is marked with the geolocation data.

16. The event server according to claim 11, wherein the steps further comprise:
retrieving the stored features from the database and splitting data from the stored features into training data and test data;
evaluating the training data for errors and coefficients;
applying machine learning algorithms to the training data; and
determining a score from the test data and machine learning algorithm results of the training data.

17. A non-transitory computer-readable medium storing instructions, that when executed by a processor, cause the processor to perform steps comprising:
receiving raw data logs from a plurality of user devices, a respective raw data log corresponding to a respective user device;
preprocessing datasets from each raw data log;
extracting dominant features for each dataset;
comparatively analyzing the datasets across raw data logs to determine common features in the datasets; and
storing the dominant and common features.

18. The non-transitory computer-readable medium according to claim 17, wherein each raw data log comprises:
heartrate measurements, geolocation data, and body temperature measurements.

19. The non-transitory computer-readable medium according to claim 17, wherein preprocessing the datasets comprises performing one or more of:
data cleansing and substitution.

20. The non-transitory computer-readable medium according to claim 17, wherein the steps further comprise:
retrieving the stored features from the database and splitting data from the stored features into training data and test data;
evaluating the training data for errors and coefficients;
applying machine learning algorithms to the training data; and
determining a score from the test data and machine learning algorithm results of the training data.
